(19) United States
(12) Patent Application Publication
(10) Pub. No.: US 2003/0158171 A1
(43) Pub. Date: Aug. 21, 2003

(54) Chelates and complexes for reduction in alcohol dependency

(75) Inventors: H. DeWayne Ashmead, Fruit Heights, UT (US); R. Charles Thompson, Peterson, UT (US)

Correspondence Address: M. Wayne Western, THORPE NORTH & WESTERN, L.L.P., P.O. Box 1219, Sandy, UT, 84091-1219, US

(73) Assignee: Albion International, inc.

(21) Appl. No.: 10/306,711

(22) Filed: Nov. 26, 2002

Related U.S. Application Data
(60) Provisional application No. 60/334,051, filed on Nov. 28, 2001.

(51) Int. Cl. A61K 31/555; A61K 31/51; A61K 31/28; A61K 31/315; A61K 31/30

(52) U.S. Cl. 514/184; 514/276; 514/494; 514/499; 514/492

(57) ABSTRACT

A method for reducing alcohol desire or dependency in a human can comprise the steps of administering a chelate or a combination of chelates to a human having alcohol dependency symptoms or an unwanted desire for alcohol. Ligands that can be used include carnitine, naturally occurring amino acids, and various thiamine molecules. Metals that can be used include nutritionally relevant metals, including copper, zinc, and manganese, to name a few.

The present application claims the benefit of U.S. Provisional Application No. 60/334,051 filed on Nov. 28, 2001, which is incorporated herein by reference.

### FIELD OF THE INVENTION

The present invention is drawn to compositions and methods for reducing alcohol dependency. More specifically, the present invention is drawn to the use of certain chelates and complexes, or combination of chelates and complexes that can be used to reduce a dependency or desire for consumption of alcohol in humans.

### BACKGROUND OF THE INVENTION

Amino acid chelates are generally produced by the reaction between α-amino acids and metal ions having a valence of two or more to form a ring structure. In such a reaction, the positive electrical charge of the metal ion can be neutralized by the electrons available through the carboxylate or free amino groups of the α-amino acid.

Traditionally, the term “chelate” has been loosely defined as a combination of a metallic ion bonded to one or more ligands to form a heterocyclic ring structure. Under this definition, chelate formation through neutralization of the positive charge(s) of the metal ion may be through the formation of ionic, covalent or coordinate covalent bonding. An alternative and more modern definition of the term “chelate” requires that the metal ion be bonded to the ligand solely by coordinate covalent bonds forming a heterocyclic ring. In either case, both are definitions that describe a metal ion and a ligand forming a heterocyclic ring.

Chelation can be confirmed and differentiated from mixtures of components by infrared spectra through comparison of the stretching of bonds or shifting of absorption caused by bond formation. As applied in the field of mineral nutrition, there are certain “chelated” products that are commercially utilized. The first is referred to as a “metal proteinate.” The American Association of Feed Control officials (AAFCO) has defined a “metal proteinate” as the product resulting from the chelation of a soluble salt with amino acids and/or partially hydrolyzed protein. Such products are referred to as the specific metal proteinate, e.g., copper proteinate, zinc proteinate, etc. Sometimes, metal proteinates are even referred to as amino acid chelates, though this characterization is not completely accurate.

The second product, referred to as an “amino acid chelate,” when properly formed, is a stable product having one or more five-membered rings formed by a reaction between the amino acid and the metal. The American Association of Feed Control Officials (AAFCO) have also issued a definition for amino acid chelates. It is officially defined as the product resulting from the reaction of a metal ion from a soluble metal salt with amino acids having a mole ratio of one mole of metal to one to three (preferably two) moles of amino acids to form coordinate covalent bonds. The average Weight of the hydrolyzed amino acids must be approximately 150 and the resulting molecular weight of the chelate must not exceed 800. The products are identified by the specific metal forming the chelate, e.g., iron amino acid chelate, copper amino acid chelate, etc.

In further detail with respect to amino acid chelates, the carboxyl oxygen and the α-amino group of the amino acid each bond with the metal ion. Such a five-membered ring is defined by the metal atom, the carboxyl oxygen, the carbonyl carbon, the α-carbon and the α-amino nitrogen. The actual structure will depend upon the ligand to metal mole ratio and whether the carboxyl oxygen forms a coordinate covalent bond or an ionic bond with the metal ion. Generally, the ligand to metal molar ratio is at least 1:1 and is preferably 2:1 or 3:1. However, in certain instances, the ratio may be 4:1. Most typically, an amino acid chelate with a divalent metal can be represented at a ligand to metal molar ratio of 2:1 according to Formula 1 as follows:

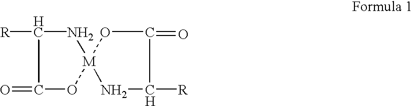

In the above formula, the dashed lines represent coordinate covalent bonds, covalent bonds, or ionic bonds. Further, when R is H, the amino acid is glycine, which is the simplest of the α-amino acids. However, R could be representative of any other side chain that, when taken in combination with the rest of the ligand structure(s), results in any of the other twenty or so naturally occurring amino acids derived from proteins. All of the amino acids have the same configuration for the positioning of the carboxyl oxygen and the α-amino nitrogen with respect to the metal ion. In other words, the chelate ring is defined by the same atoms in each instance, even though the R side chain group may vary.

With respect to both amino acid chelates and metal proteinates, the reason a metal atom can accept bonds over and above the oxidation state of the metal is due to the nature of chelation. For example, at the α-amino group of an amino acid, the nitrogen contributes to both of the electrons used in the bonding. These electrons fill available spaces in the d-orbitals forming a coordinate covalent bond. Thus, a metal ion with a normal valency of +2 can be bonded by four bonds when fully chelated. In this state, the chelate is completely satisfied by the bonding electrons and the charge on the metal atom (as well as on the overall molecule) is zero. As stated previously, it is possible that the metal ion can be bonded to the carboxyl oxygen by either coordinate covalent bonds or ionic bonds. However, the metal ion is preferably bonded to the α-amino group by coordinate covalent bonds only.

The structure, chemistry, bioavailability, and various applications of amino acid chelates are well documented in the literature, e.g. Ashmead et al., Chelated Mineral Nutrition, (1982), Chas. C. Thomas Publishers, Springfield, Ill.; Ashmead et al., Intestinal Absorption of Metal Ions, (1985), Chas. C. Thomas Publishers, Springfield, Ill.; Ashmead et al., Foliar Feeding of Plants with Amino Acid Chelates, (1986), Noyes Publications, Park Ridge, N.J.; U.S. Pat. Nos. 4,020,158; 4,167,564; 4,216,143; 4,216,144; 4,599,152; 4,725,427; 4,774,089; 4,830,716; 4,863,898; 5,292,538; 5,292,729; 5,516,925; 5,596,016; 5,882,685; 6,159,530; 6,166,071; 6,207,204; 6,294,207; 6,614,553; each of which are incorporated herein by reference.

One advantage of amino acid chelates in the field of mineral nutrition is attributed to the fact that these chelates are readily absorbed from the gut and into mucosal cells by means of active transport. In other words, the minerals can be absorbed along with the amino acids as a single unit utilizing the amino acids as carrier molecules. Therefore, the problems associated with the competition of ions for active sites and the suppression of specific nutritive mineral elements by others can be avoided.

Thiamine, also known as thiamin and vitamin B₁, is a water-soluble vitamin that functions as part of several enzymes (thiamine pyrophosphate, thiamine di-phosphate, or TPP, which is thiamine and two molecules of phosphoric acid) essential for energy production, carbohydrate metabolism, and nerve function. The following is a structure for thiamine, shown as Formula 2 below:

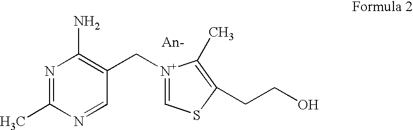

In the above formula, An⁻ can be any ion that counterbalances the positive charge on the thiazole nitrogen. For example, An⁻ can be NO₃⁻, Cl⁻, or another counter-ion. The structure of thiamine di-phosphate, one of the bioactive forms of thiamine, is shown below as Formula 3:

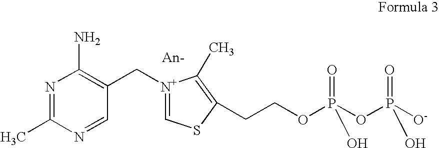

In addition to thiamine di-phosphates, thiamine mono-phosphate and thiamine tri-phosphate are also known to be important with regard to function in humans.

Some clinical studies have shown that thiamine supplementation may be helpful in alcoholic patients, Alzheimer's patients, in cases of senility (mental impairment) associated with the elderly, and in epileptics being treated with Dilatin. Thiamine is known to be rapidly absorbed in the upper and lower small intestine, and further, is transported by the circulatory system to the heart, liver, kidneys, and other organs. At these sites, thiamine can be combined with magnesium and/or specific proteins to become active enzymes for the metabolizing of carbohydrates into simple sugars. However, thiamine is not stored in the body in any great quantity, and is excreted in the urine. Therefore, in order to benefit from the presence of thiamine in the body, it is preferred that it be taken in, usually orally, on a daily basis. Alcohol interferes with the absorption of all nutrients, but especially Thiamine (vitamin B₁) and riboflavin (Vitamin B₂). In fact, thiamine deficiency can affect every cell in the body.

Alcoholics and binge drinkers are especially prone to thiamine deficiency as alcohol can reduce absorption, alter metabolism, and deplete body stores. Further, alcoholics also tend to have poor diets. Thiamine deficiency is also associated with some of the symptoms of alcoholism such as mental confusion and visual disturbances. If thiamine deficiency is not corrected, permanent brain damage may result. This condition is known as Wemicke Korsakoff syndrome and is usually seen in people who have been addicted to alcohol for many years.

### SUMMARY OF THE INVENTION

It has been recognized that the use of certain chelates and chelate complexes can reduce alcohol desire and/or dependency in humans. In one embodiment, a method for reducing alcohol desire or dependency in a human can comprise the steps of administering an amino acid chelate or a combination of amino acid chelate to a human having alcohol dependency symptoms or an unwanted desire for alcohol, wherein the amino acid chelate(s) comprises a naturally occurring amino acid ligand and a metal selected from the group consisting of copper, zinc, and manganese, and wherein the amino acid to metal molar ratio is from 1:1 to 4:1.

Additionally, a composition for reducing alcohol dependency in humans can comprise a particulate blend of a first amino acid chelate and a second amino acid chelate, wherein the first amino acid chelate and the second amino acid chelate each comprise a different metal selected from the group consisting of copper, zinc, and manganese.

An alternative composition and method for reducing alcohol desire or dependency is also provided. The composition can comprise a naturally occurring amino acid chelated to a nutritionally relevant metal, wherein the composition further comprises thiamine complexed to the nutritionally relevant metal. An associated method can comprise the step of administering the composition to a human to reduce alcohol desire or dependency. Bioactive forms of thiamine can also be complexed to the metal, such as for example, thiamine phosphates including thiamine mono-phosphate, thiamine di-phosphate, and thiamine tri-phosphate. In either case, typically, the amino acid is chelated to the metal at the carboxyl oxygen and the α-amino nitrogen. Additionally, the thiamine can be complexed to the metal at the N¹ position of the pyrimidine ring, or in the case of the thiamine phosphates, complexation can also occur at a phosphate moiety of the ligand.

In an alternative embodiment, carnitine can be used as a ligand (rather than a traditional amino acid) to chelate copper, zinc, or manganese. Though carnitine is not an amino acid in the traditional sense, i.e., used for forming the basic constituents of proteins, it does contain a tertiary amino group and an acid group. As such, carnitine can be a good ligand for use in accordance with the present invention. More specifically, a composition and method for reducing alcohol desire or dependency is provided. The composition can comprise a metal carnitine chelate comprising a metal selected from the group consisting of copper, zinc, or manganese, and wherein the carnitine to metal molar ratio is from 1:1 to 2:1. An associate method can comprise the step of administering to a human a therapeutically effective amount of the metal carnitine chelate.

In another embodiment, thiamine chelates can also be administered to reduce alcohol dependency in humans.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

It is to be noted that, as used in this specification and the appended claims, the singular forms “a,” “an,” and “the” include plural referents unless the context clearly dictates otherwise.

The term “naturally occurring amino acid” or “traditional amino acid” shall mean amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

The term “amino acid chelate” is intended to cover both the traditional definitions and the more modem definition of chelate as cited previously. Specifically, with respect to chelates that utilize traditional amino acid ligands, i.e., those used in forming proteins, chelate is meant to include metal ions bonded to proteinaceous ligands forming heterocyclic rings. Between the carboxyl oxygen and the metal, the bond can covalent or ionic, but is preferably coordinate covalent. Additionally, at the α-amino group, the bond is typically a coordinate covalent bond. Proteinates of naturally occurring amino acids are included in this definition.

The term “proteinate” when referring to an iron proteinate is meant to include compounds where iron is chelated or complexed to hydrolyzed or partially hydrolyzed protein forming a heterocyclic ring. Coordinate covalent bonds, covalent bonds and/or ionic bonding may be present in the chelate or chelate/complex structure. As used herein, proteinates are included when referring to amino acid chelates.

“Amino acid thiamine chelate complex” or “thiamine-complexed metal amino acid chelate” can include compositions comprising a metal, an amino acid, and a thiamine molecule. The amino acid is typically chelated to the metal as described under the “amino acid chelate” definition, and the thiamine ligand is typically complexed to the metal, such as at the N₁ position of the pyrimidine ring or at a phosphate group when a thiamine phosphate is used. Other molecules can also be complexed to or coordinated with the metal including water, acids, nitrates, or chlorides.

The term “nutritionally relevant metal” is meant to mean any divalent (and in some embodiments, trivalent) metal that can be used as part of a nutritional supplement, is known to be beneficial to humans, and is substantially non-toxic when administered in traditional amounts, as is known in the art. Examples of such metals include copper, zinc, manganese, iron, chromium, cobalt, calcium, and the like.

With these definitions in mind, various methods and compositions are disclosed herein that are beneficial in reducing alcohol dependency and/or desire in humans.

Amino Acid Chelates for Reduction of Alcohol Desire or Dependency

A method for reducing alcohol desire or physical dependency in a human can comprise the steps of administering an amino acid chelate to a human having alcohol dependency symptoms or an unwanted desire for alcohol, wherein the amino acid chelate comprises a naturally occurring amino acid ligand and a metal selected from the group consisting of copper, zinc, and manganese, and wherein the amino acid to metal molar ratio is from 1:1 to 4:1. Metal proteinates having any one of the same metals can also be used.

Additionally, a composition for reducing alcohol dependency in humans can comprise a particulate blend of a first amino acid chelate and a second amino acid chelate, wherein the first amino acid chelate and the second amino acid chelate each comprise a different metal selected from the group consisting of copper, zinc, and manganese. In a further detailed aspect of an embodiment of the invention, three metal amino acid chelates can be present in the particulate blend, wherein one comprises zinc, a second comprises copper, and a third comprises manganese.

With respect to both the method and composition embodiments, the naturally occurring amino acid ligand can be selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof, and dipeptides, tripeptides, and tetrapeptides formed by any combination of said amino acids thereof In a more detailed aspect, though any of the above traditional amino acid ligands can be used effectively, amino acid ligands having anti-oxidant properties or other alcohol dependency reduction properties can be preferred for use in some embodiments. Such amino acids include those selected from the group consisting of cystine, cysteine, arginine, histidine, lysine, glycine, and glutamic acid, and combinations thereof.

Regarding the method embodiment described, one of copper, zinc, or manganese can be used as the sole metal selected for use having one or more amino acid ligands chelated to the single metal. Alternatively, a combination of at least two metals can be present in an amino acid chelate cocktail formulation. Thus, any two of copper, zinc, and manganese can be selected for use. Again, with this embodiment, one or more ligand(s) can be independently chelated to each of the metals. In yet another embodiment, all three metals can be present in a composition mixture including a copper amino acid chelate, a zinc amino acid chelate, and a manganese amino acid chelate. Again, one or more ligands can be used to chelate the metals.

In embodiments where multiple amino acid chelates are present as part of a liquid or particulate dosage, or which are mixed with a carrier, the amino acid chelates present can be divided into various ratios. For example, if a zinc amino acid chelate is mixed with a copper amino acid chelate, of the total amino acid chelate present, the copper amino acid chelate to zinc amino acid chelate can be present at a weight ratio from about 1:40 to 40:1, with a preferred range being from about 5:1 to 1:5. The same ratios would also be present in embodiments where other amino acid chelates are prepared in a mixed batch, i.e., zinc/manganese or copper/manganese. When all three metals are present as amino acid chelates, then each metal amino acid chelate compared to the remaining two metal amino acid chelates should be present at a weight ratio of at least 1:20, e.g., zinc amino acid chelate to copper and manganese amino acid chelates should be at least 1:20. These ratios are given with respect to the presence of certain amino acid chelates without regard to weight variation of various amino acid ligands. With each embodiment, as stated previously, one or more amino acid ligands can be used for the amino acid chelates described. However, since the ratios given are by weight, one can consider the molecular weight of each amino acid ligand, as well as the molecular weight of the metals, when determining appropriate weight ratios.

It is not the purpose of the present invention to describe how to prepare amino acid chelates that can be used with the present invention. Any amino acid chelate or combination of chelates comprising copper, zinc, and/or manganese can be used with varying degrees of effectiveness. Suitable methods for preparing such amino acid chelates can include those described in U.S. Pat. No. 4,830,716, for example. However, combinations of such chelates as part of a particulate composition for reducing alcoholic desire and/or dependency are included as an embodiment of the present invention.

One reason that copper, zinc, and manganese are selected as metals for use to reduce alcohol desire and/or alcohol dependency in humans and other mammals is due to the fact that ethanol abuse is believed to exacerbate deficiencies of these metals, particularly with respect to copper and zinc. As ethanol metabolism becomes altered, frequently, an increase in alcohol intake results, whether by merely increased desire or dependency. Additionally, with respect to embodiments where one or more of the amino acid ligands used has anti-oxidant properties, the presence of free radicals in the body associated with alcohol metabolism and subsequent alcohol dependency can be reduced. This being said, the purpose of the invention is not to describe any specific mechanism as to why these compositions reduce alcohol desire or dependency in humans, only that the compositions and methods that are in accordance with an embodiment of the present invention reduce such alcohol desire or dependency.

Carnitine Chelates for Reduction of Alcohol Desire or Dependency

In an alternative embodiment, carnitine can be used as a chelating ligand for copper, zinc, or manganese, and this composition can be effective in reducing alcohol desire and/or dependency. Carnitine comprises an acid group and a tertiary amino group, and has the following structure:

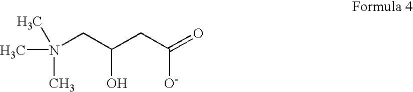

Carnitine is not an amino acid in the traditional sense, i.e., used for forming the basic constituents of proteins. However, it does contain a tertiary amino group and an acid group. Therefore, in a technical sense, it is an amino acid. Carnitine can also be a good ligand for use in accordance with the present invention, even though it does not chelate metals by the same mechanism as traditional amino acids.

Carnitine is a somewhat unique molecule in that it has a methylated tertiary nitrogen that carries a fixed positive charge at its tertiary amine group. The tertiary nitrogen is balanced by an equal negative charge on the carboxylate group of the molecule. In this configuration, the molecule is said to be “zwitterionic,” because of two full opposite charges carried by the molecule. A somewhat unique characteristic of carnitine comes from the fact that it exists in this zwittwerionic form, regardless of pH. All of the naturally occurring or traditional amino acids can form zwitterions, but the ionization of most amino acids is dependant on the pH of the solution in which they are dissolved. Carnitine exists as a zwitterion independent of the solution pH.

When the carboxylate group of a naturally occurring amino acid loses its hydrogen, or is ionized, the negative charge present can be shared by both of the oxygen atoms of the carboxylate group. There are two possible resonance structures for an ionized carboxylate group, where one or the other oxygen carries the negative charge created by the removal of the carboxylate proton. In reality the charge resonates back and forth between the oxygen atoms creating a structure where the two oxygen molecules share the negative charge. With respect to carnitine, the “fixed” zwitterionic charge on carnitine has a full positive charge on the nitrogen, and a resonance or shared full negative charge on the carboxylate group.

There are four carnitine chelate formulations that are contemplated by the present invention, including two 1:1 carnitine to metal chelates, and two 2:1 carnitine to metal chelates, as shown below in Formulas 5 and 6.

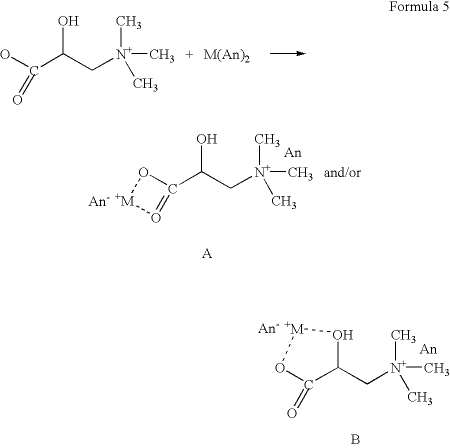

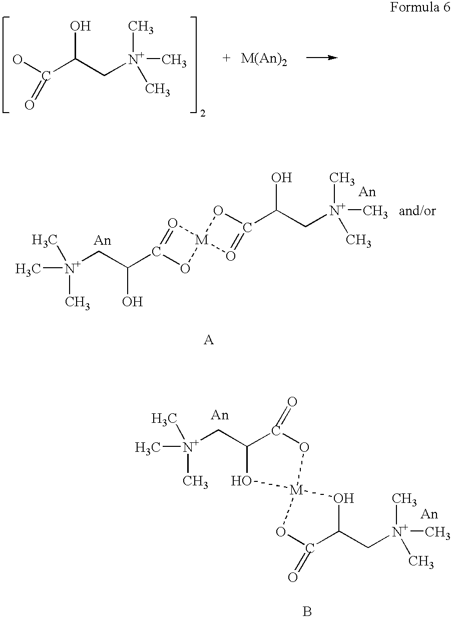

In both Formula 5 and 6 above, M can be Cu, Zn, or Mn, and An can be Cl, NO₃, or acetate. Other metals (M) and other anions (An⁻) can be used as would be apparent to one skilled in the art after considering the present disclosure.

As shown in Formulas 5 and 6 above, a metal ion can be chelated to the carnitine ligand in one of two ways. In one embodiment, the metal can be bound only to the carboxylate end of one carnitine molecule in a 1:1 carnitine to metal molar ratio embodiment (Structure A of Formula 5) or to the carboxylate end of two carnitine molecules in a 2:1 carnitine to metal molar ratio embodiment (Structure A of Formula 6). This type of bonding is common for ionized carboxylate groups, and still meets a broader definition of a chelate as it provides a ring structure. Though a double bond is shown at the carboxylate group, in actuality, charge sharing occurs between the two oxygen atoms and a true double bond does not exist.

Alternatively, the negative charge on the carboxylate group can be fixed to one of its oxygen atoms as a result of chelation to the metal ion. The hydroxyl group on the carbon-2 atom of carnitine can also participates in the chelation bonding of carnitine to the metal ion, creating a more traditional metal ligand chelate. Even though the hydroxyl group is still shown to maintain its hydrogen atom upon chelation, a ring structure can still be formed by attraction between the metal and the hydroxyl group. A 1:1 carnitine to metal molar ratio (Structure B of Formula 5) or a 2:1 carnitine to metal molar ratio (Structure B of Formula 6) can be formed.

With respect to Structure A of Formula 5 and Structure A of Formula 6, even after chelation, the charge on the metal (M) is still not satisfied, and therefore, can form a salt with the anion (An⁻) present in the reaction. In other words, when the carboxylate group of carnitine is bonded to a metal ion, the positive charge also needs to be balanced by an equal negative charge. This can be achieved by using a metal salt that dissociates and balances the charge on both the tertiary amine of the carnitine and the metal. This is not necessary with the 2:1 embodiments, as the metal charges can be balance by the second carnitine ligand, if the metal is divalent. If the metal is trivalent and the ligand to metal molar ratio is 2:1, the metal (M) can be counter-balanced by an anion (An⁻) similar to that shown in the 1:1 embodiment.

In accordance with methods of the present invention, carnitine chelates can be used alone to reduce alcohol dependency. As mentioned, with carnitine, chelation can occur at the hydroxyl group and the carboxyl group to form a 5 membered ring, or alternative, at the carboxyl group alone to form a 4 membered ring (charge is shared between both oxygen molecules of the carboxyl group). In either embodiment, as long as a chelate is formed, the composition and method is within the scope of the present invention, and is considered to be a metal carnitine chelate.

Alternatively, it may be desired to coadminister other components that aid in the alcohol dependency reduction. As such, an amino acid chelate can be admixed or blended with the metal carnitine chelate, wherein the amino acid chelate comprises a naturally occurring amino acid ligand and a metal selected from the group consisting of copper, zinc, and manganese, and wherein the amino acid to metal molar ratio is from 1:1 to 4:1. Thiamine or a thiamine-containing composition can also be admixed with the carnitine chelates of the present invention. These admixtures can be present at from 40:1 to 1:40 by weight.

Thiamine-Containing Chelates for Reduction of Alcohol Desire or Dependency

Thiamine is a composition that is effective in reducing alcohol desire or dependency in humans. Thus, thiamine, thiamine phosphates, thiamine hydrochloride, thiamine mononitrate, or other known thiamine salts can be coadministered with one or more of the other compositions disclosed herein that also reduce such a desire or dependency. Alternatively, a thiamine-containing chelate composition can also be administered in accordance with the present invention. Examples of thiamine-containing chelate compositions that can be administered alone (or in combination with other compositions) to reduce alcohol dependency include thiamine-complexed metal amino acid chelates and/or π-bond aromatic thiamine chelates.

Thus, in accordance with embodiments of the present invention, a thiamine-complexed metal amino acid chelate is provided herein that can comprise a nutritionally relevant metal; a naturally occurring amino acid chelated to the metal; and a thiamine complexed to the metal. Any bioactive form of thiamine can be used including, but not limited to thiamine hydrochloride and thiamine mononitrate. Additionally, more bioactive forms of thiamine can also be complexed to the metal, such as for example, thiamine phosphates including thiamine mono-phosphate, thiamine di-phosphate, and thiamine tri-phosphate. As mentioned, this composition can be administered to a human to reduce alcohol desire and/or dependency.

With respect to the thiamine-complexed metal amino acid chelates per se, an amino acid is always present. In one embodiment, the amino acid can be chelated to the metal. This will normally occur at the carboxyl oxygen and the α-amino nitrogen, as described previously. Further, if the amino acid is chelated to the metal as described, the thiamine can be complexed to the metal. In one embodiment, the complexing between the metal and the thiamine ligand will be at the pyrimidine ring, preferably at N¹ (the first position nitrogen) of the pyrimidine ring. However, this is not required. For example, in the case of thiamine phosphate embodiments, the complexation can occur at a phosphate moiety. In other words, all that is required with this embodiment is that a chelate complex exist.

In the present embodiment, the amino acid to metal molar ratio can be from 1:1 to 2:1, and even 3:1 if the amino acid is one of the smaller amino acids, e.g., glycine. The naturally occurring amino acids that can be selected for use include those selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof, and dipeptides, tripeptides, and tetrapeptides formed by any combination of the above amino acids thereof. In a more detailed aspect, though any of the above amino acid ligands can be used effectively, amino acid ligands having anti-oxidant properties can be preferred for use in some embodiments. Such amino acids include those selected from the group consisting of cystine, cysteine, arginine, histidine, lysine, glutamic acid, and combinations thereof. Another preferred amino acid for use is glycine.

With respect to the amount of thiamine present in the thiamine-complexed metal amino acid chelates of the present invention, a thiamine to metal molar ratio from about 1:1 to 2:1 can be present. As thiamine is a relatively large molecule, the presence of more than two ligands on a single metal ion in this embodiment can be more difficult to achieve, as an amino acid will typically be chelated to the metal ion as well. Thus, in a preferred embodiment, the thiamine to amino acid to metal molar ratio can be about 1:1:1, though 2:1:1 or 1:2:1 can also be used to reduce alcohol dependency.

A mixture of these compositions is also within the scope of the present invention. In embodiments where multiple amino acid thiamine chelate complexes are present as part of a liquid or particulate dosage, or which are mixed with a carrier, the amino acid thiamine chelate complexes present can be divided into various ratios. For example, a mixture of amino acid thiamine chelate complexes can be prepared by formulating a zinc amino acid thiamine chelate complex and a copper amino acid thiamine chelate complex for administration together. Further, manganese amino acid thiamine chelate complexes can be admixed with one or both of the zinc or copper chelate complexes.

Though not required, in one embodiment, the coordination number of the metal can be configured to be fully satisfied. Table 1 below lists the coordination numbers for three metal ions that can be used in accordance with an embodiment of the present invention.

TABLE 1

| Metal Ion | Coordination Number |
|---|---|
| Cu(I) | 2,4 |
| Cu(II) | 4,6 |
| Mn(II) | 6 |
| Mn(III) | 6 |
| Zn(II) | 4,6 |

In each of the above, the coordination number may be satisfied with the amino acid, the thiamine molecule, or with another polydentate or unidentate ligand. For example, a metal amino acid thiamine chelate complex having a 1:1:1 molar ratio can satisfy the copper(II) coordination number of four by the polydentate amino acid ligand satisfying two sites, the thiamine satisfying one site, and an ancillary molecule such as water, chloride, or organic acid satisfying one site with a coordination bond. An example of such a structure is provided below as Formula 7:

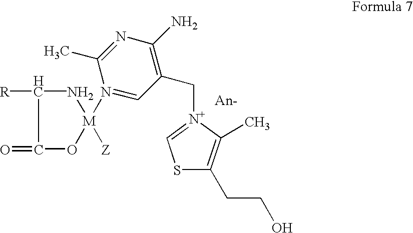

In the above formula, An⁻ can be any anion that counterbalances the positive charge on the thiazole nitrogen; M can be any nutritionally relevant metal (including copper, zinc, and manganese); Z can be an ancillary molecule that can optionally be present to satisfy a coordination number of metal; R can be representative of any side chain that, when taken in combination with the rest of the amino acid ligand structure, results in one of the twenty or so naturally occurring amino acids derived from proteins. In some embodiments, Z can be an acid that acts to fulfill the coordination site of other adjacent amino acid thiamine chelate complexes as well, as exemplified in some of the examples provided herein. Formula 7 is given by way of example only, and can be modified in accordance with embodiments of principles of the invention. For example, two thiamine molecules and/or two amino acids can be present. Alternatively, thiamine phosphates can be used instead of or with other thiamine molecules. If it is desired to fulfill a metal coordination number of six, then additional ligands can be used to achieve such a coordination number, e.g., two amino acids fulfilling four coordination sites, one thiamine fulfilling one coordination site, and one organic acid fulfilling one coordination site. Thus, the only limitations as to the number of ligands that can be present is provided by the coordination number of the metal ion, and the stereo chemistry which may prohibit a combination of ligands to be bound to a common metal ion based on size, stereochemistry, or number.

In accordance with an alternative embodiment, a method for reducing alcohol desire or dependency in a human can comprise the step of administering a thiamine-complexed metal amino acid chelate described herein to a human having alcohol dependency symptoms or an unwanted desire for alcohol. As described, the amino acid chelate complex can comprise at least one naturally occurring amino acid ligand, at least one thiamine ligand, and at least one metal selected from the group consisting of copper, zinc, and manganese. The ligand to metal molar ratio can be from 1:1 to 4:1. Alternatively, proteinates can be used as a ligand in accordance with an embodiment of the invention.

As described previously with respect to the metal amino acid chelates, one reason that copper, zinc, and manganese are preferred metals for use to reduce alcohol desire and/or alcohol dependency in humans and other mammals is due to the fact that ethanol abuse is believed to exacerbate deficiencies of these metals, particularly with respect to copper and zinc. As ethanol metabolism becomes altered, frequently, an increase in alcohol intake results, whether by merely increased desire or by increased dependency. However, because the ligand thiamine of itself has properties that reduce a desire and/or dependency for alcohol, these three metals are not required for use when thiamine is present. Any nutritionally relevant metal can be used including calcium, magnesium, iron, and other minerals that can form a chelate complex in accordance with embodiments of the present invention. Additionally, with respect to embodiments where one or more of the amino acid ligands used has anti-oxidant properties, the presence of free radicals in the body associated with alcohol dependency can be reduced. This being said, the purpose of the invention is not to describe any specific mechanism as to why these compositions reduce alcohol desire or dependency in humans, only that the methods of the present invention reduce alcohol desire or dependency.

Other thiamine-containing compositions, including thiamine chelates, can also be used to reduce alcohol desire and/or dependency in humans, such as those described in U.S. Pat. No. 5,292,729. Formula 8 below provides such an example:

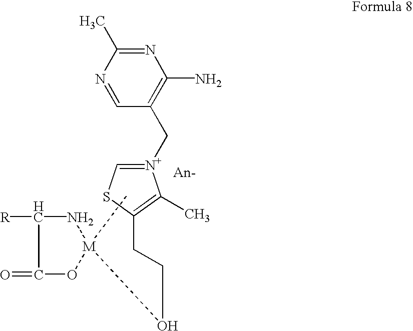

In the above formula, An⁻ can be any ion that counterbalances the positive charge on the thiazole nitrogen; M can be a metal selected from the group consisting of copper, zinc, and manganese; R can be representative of any side chain that, when taken in combination with the rest of the amino acid ligand structure, results in one of the twenty or so naturally occurring amino acids derived from proteins. In Formula 8 above, instead of an electronegative atom forming a coordinate-covalent bond, that bond can be replaced by a bond formed between the metal ion and a π-cloud of the aromatic ring of the thiamine ligand. Because π-clouds are rich in electrons, they can behave as nucleophiles. When π-cloud types of chelates are formed between the aromatic ring of a water-soluble thiamine ligand and a metal ion, greater stability and enhanced absorption and utilization (i.e. increased bioavailability) can be observed for both the metal and the thiamine ligand. In one embodiment, the lone pair of electrons on an oxygen atom pendent to the thiazole aromatic ring can serve as one electron pair and the π-cloud on the thiazole aromatic ring can serve as the other electron pair. Again, Formula 8 is provided by way of example, as other arrangements of thiamine and amino acids with respect to the metal are possible, as would be ascertainable by one skilled in the art after reading the present disclosure.

Combinations of Compositions Effective for Reduction of Alcohol Desire or Dependency

Various compositions and methods are provided herein that can be used to reduce desire or dependency for alcohol. Specifically, as disclosed herein, effective compositions that can be used include: 1) amino acid chelates comprising copper, zinc, or manganese; 2) thiamine chelates comprising any nutritionally relevant metal; 3) amino acid thiamine chelate complexes comprising any nutritionally relevant metal; and 4) carnitine chelates comprising copper, zinc, or manganese. However, it is important to note that these compositions can be administered in any combination to provide a desired affect. Additionally, other compositions can be coadministered with the above compositions to enhance a therapeutic result, including copper complexes, zinc complexes, manganese complexes, thiamine, thiamine hydrochloride, thiamine mononitrate, thiamine phosphates, amino acids with antioxidant properties, etc. Any combination of the above compositions can be coadministered at a weight ratio from about 40:1 to 1:40.

### EXAMPLES

The following examples are illustrative of a present method of reducing alcohol dependency in humans, as well as compositions that can be used for the same. As such, the following examples should not be considered as limitations of the present invention, but merely demonstrate the effectiveness of the methods and compositions described herein.

### Example 1

### Reduction of Alcohol Dependency in Laboratory Rats

Preparative Procedures and Conditions

Ten male Sprague Dawley albino rats of similar age and weight were individually caged in an environment maintained at 20° C. The rats were randomly separated into a control group and a treatment group (5 rats in each group). All rats were fed dry laboratory rat chow ad libitum, and the basal diet of each rat contained 20 ppm zinc carbonate, 15 ppm copper carbonate, and other essential nutrients. Additionally, each rat cage was fitted with two bottles, one containing water, and the other containing a mixture of 5% ethanol and 95% water (v/v). All of the water used was distilled and deionized. The positions of the water bottles on each cage were changed daily to reduce the likelihood of each rat learning the locations of the different bottles, and thus, influence the selection of which bottle the rat chose to drink from. All rats had access to both the water and the ethanol/water bottle throughout the entire study ad libitum.

Phase 1—Developing Alcohol Dependency in Rats

All of the rats were intragastrically administered a 1:1 volume of water and ethanol in an amount such that 8 g of ethanol per kg of body weight was received. This amount is equivalent to about ⅔ of the lethal dose for rats. Such a dosage was administered every morning at the same time for a 28 day period, which created a physical need for ethanol in each of the rats. See, Liubimov B I et al., Chronic alcoholic intoxications in animals or a model for studying the safety of new anti-alcoholic agents (abstract), Farmakol Toksiol 46:98-102, Physiological Abstract 9010 (1983). Each rat was weighed once a week on an electronic balance and the daily quantity of administered ethanol was adjusted according to each rat's individual weekly weight. Because all of the rats were approximately the same size, each received approximately 2.2 ml of ethanol per day.

Phase 2—Study of Effects of Amino Acid Chelates on Alcohol Dependency in Rats

After 28 days (Phase 1), the intragastric administration of ethanol was discontinued with all of the rats, both from the control and treatment group. While familiar to the ethanol/water dispensing bottle and the water-dispensing bottle described previously, each rat had to seek out the ethanol/water-dispensing bottle as its sole source of ethanol.

At day 29, each of the 5 rats in the treated group received a daily dose of zinc, manganese, and copper amino acid chelates dissolved in distilled and deionized water. About 100 μg (approximately 0.49mg/kg body wt) of zinc, 100 μg of manganese (approximately 0.49 mg/kg body wt) and 2 μg copper (approximately 0.01 mg/kg body wt) was present in the water. The diluted mineral solution was administered daily to each rat in the treated group intragastrically using a similar method described in phase 1. This phase continued for a total of 21 days.

Regarding the control group, even though they received intrinsic copper, manganese, and zinc salts as part of the rat chow formulation, they did not receive any copper, manganese, or zinc amino acid chelates during any phase of the study.

With respect to both the control group and the treatment group, beginning on day 29, the quantities of both the ethanol/water mixture (Et-OH/H₂O) and the water (H₂O) consumed in each 24 hour period were measured and recorded, as is shown in Table 2 below:

TABLE 2

| | CONTROL GROUP | | TREATED GROUP | |
|---|---|---|---|---|
| Day | H₂O | Et-OH/H₂O | H₂O | Et-OH/H₂O |
| (phase 2) | (ml/rat) | (ml/rat) | (ml/rat) | (ml/rat) |
| 29 | 0 | 0 | 0 | 0 |
| 30 | 43.0 | 8.3 | 50 | 0 |
| 31 | 21.3 | 14.0 | 35.3 | 1.5 |
| 32 | 8.3 | 7.0 | 20.0 | 0.75 |
| 33 | 9.3 | 7.6 | 25.0 | 1.0 |
| 34 | 7.2 | 7.1 | 18.0 | 0.75 |
| 35 | 3.2 | 9.0 | 36.5 | 1.25 |
| 36 | 10.2 | 7.2 | 33.0 | 1.0 |
| 37 | 15.0 | 10.3 | 25.0 | 1.0 |
| 38 | 10.2 | 13.0 | 35.5 | 1.0 |
| 39 | 15.5 | 22.0 | 45.5 | 1.0 |
| 40 | 14.5 | 14.0 | 29.0 | 1.0 |
| 41 | 22.5 | 11.0 | 41.0 | 0.5 |
| 42 | 28.0 | 11.0 | 40.5 | 2.0 |
| 43 | 20.0 | 12.5 | 35.0 | 0.5 |
| 44 | 20.5 | 9.5 | 38.0 | 1.0 |
| 45 | 15.0 | 13.0 | 37.5 | 0.5 |
| 46 | 29.5 | 9.5 | 30.0 | 1.0 |
| 47 | 19.0 | 15.0 | 36.0 | 0.5 |
| 48 | 30.0 | 11.5 | 36.0 | 2.0 |
| 49 | 14.5 | 14.5 | 32.5 | 0.5 |

Table 3 below shows the mean weights and liquid consumption of the rats from both the control group and the treated group.

TABLE 3

| | CONTROL GROUP | TREATED GROUP |
|---|---|---|
| | Mean Wt/Liq Con | Mean Wt/Liq Con |
| Initial Weight | 191.1 g/21.8 g | 189.6 g/24.0 g |
| Terminal Weight | 233.8 g/24.3 g | 210.9 g/13.8 g |
| H₂O consumption/day | 12.9 ml/9.5 ml | 33.9 ml/7.9 ml |
| (Phase 2) | | |
| ethanol consumption/day | 2.3 ml/0.72 ml | 0.1 ml/0.02 ml |
| (Phase 2) | | |
| ethanol/H₂O consumption/day | 11.4 ml/3.6 ml | 0.9 ml/0.5 ml |
| (Phase 2) | | |
| total liquid consumption/day | 24.3 ml/10.3 ml | 34.8 ml/7.8 ml |
| (Phase 2) | | |

Results

By the end of Phase 1 (the first 28 day period), all of the rats in both groups exhibited behavior suggesting ethanol abuse. All animals preferred drinking from the ethanol/water bottle over the water bottle during Phase 1.

As can be seen from Tables 2 and 3, the rats that were supplemented with zinc and copper amino acid chelates (the treated group) exhibited a significantly reduced ethanol/water consumption in phase 2 as compared to the control group. Further, in phase 2, the water consumption among the treated group was much greater than among the control group. This shows that, in rats with confirmed ethanol abuse, there was significantly less desire to consume ethanol when the animals received zinc and copper as amino acid chelates.

### Example 2

### Preparation of Admixture of Copper Bisarginate and Zinc Bisarginate

An amino acid chelate containing a particulate mixture was prepared comprising about 45 μg (0.22 mg/kg body wt) of a copper bisarginate and 100 μg (0.49 mg/kg body wt) of a zinc bisarginate. This formulation, when administered in an oral dosage form, or with a carrier, provides reduced alcohol dependency and/or desire among alcohol dependent animals.

### Example 3

### Preparation of Admixture of Copper Bisglycinate, Zinc Bisglycinate, and Manganese Bisglycinate

An amino acid chelate containing a particulate mixture was prepared comprising about 20 mg of a copper bisglycinate, 30 mg of a zinc bisglycinate, and 20 mg of a manganese bisglycinate. This formulation, when administered as an oral dosage tablet or in another similar form, or with a carrier, provides reduced alcohol dependency and/or desire among alcohol dependent humans.

### Example 4

### Preparation of 1:1 Copper Carnitine Chelates

A 1:1 molar ratio of a copper carnitine chelate can be prepared by reacting 0.5 moles of a Cu(Cl)₂ (67.2 g/L) and 0.5 moles of carnitine (80.6 g/L) in an aqueous solution, as shown below:

Cu(Cl)₂+Carnitine Cl:CuCarnitine:Cl

In accordance with the above reaction scheme, the following procedures were followed to obtain about 147.8 g of total dissolved solids. Specifically, 0.5 moles (80.6 g) of carnitine was dissolved in one liter of water, and the mixture was brought to 55-60° C. Next, 0.5 moles (67.2 g) of cupric chloride was added to the mixture, and the mixture was allowed to react for a total of 4 hours. After the 4 hour reaction time, the composition was cooled 40° C. and spray dried to obtain about 147.8 g of a 1:1 copper carnitine chelate product at 100% yield.

### Example 5

### Preparation of 2:1 Copper Carnitine Chelates

A 2:1 molar ratio of a copper carnitine chelate can be prepared by reacting 0.5 moles of a Cu(Cl)₂ (67.2 g/L) and 1 moles of carnitine (161.2 g/L) in an aqueous solution, as shown below:

Cu(Cl)₂+2Carnitine Cu(Carnitine:Cl)₂

In accordance with the above reaction scheme, the following procedures were followed to obtain about 228.4 g of total dissolved solids. Specifically, 1.0 moles (161.2 g) of Carnitine was dissolved in one liter of water, and the mixture was brought to 55-60° C. Next, 0.5 moles (67.2 g) of cupric chloride was added to the mixture and allowed to react for a total of 4 hours. After 4 hours of reaction time, the composition was cooled to 40° C. and spray dry to obtain about 228.4 g of a 2:1 copper carnitine chelate at 100% yield.

### Example 6

### Preparation of a Copper Glycine Thiamine Chelate Complex

A copper amino acid thiamine chelate complex was prepared by, first, reacting an equal molar mixture of copper hydroxide (48.8 g/L Cu(OH)₂), thiamine mono-nitrate (163.7 g/L Thi⁺NO₃⁻), and glycine (37.5 g/L Gly). Next, a source of acidic protons was added to help drive off the hydroxide ions (OH⁻) of the copper hydroxide. In the present example, citric acid (Citric) was added to the mixture. The following stoichiometry is provided to illustrate the reaction:

0.5Cu(OH)₂+0.5Thi⁺NO₃⁻+0.5Gly+0.167Citric

0.5ThiNO₃⁻(Cu)Gly+0.167Citrate+H₂O

In accordance with the above reaction scheme, the following procedures were followed to obtain about 283.5 g of total dissolved solids. Specifically, to 1 liter of water was added about 163.7 g of thiamine mono-nitrate, and the solution was brought to about 40° C. to 60° C. About 48.8 g of copper hydroxide was then added and was allowed to react for 15 minutes. Next, about 38.4 g of glycine was added. After allowing the reaction to progress for another 15 minutes, about 32.1 g or citric acid was added. After about 3 hours of further reaction, the product was spray dried at 40° C. About 283.5 g of product at about a 100% yield was produced.

It is believed that the coordination number of four can be fully satisfied by this reaction, though this is not required. Specifically, two coordination sites can be satisfied by the glycine, one coordination site can be satisfied by the thiamine, and one coordination site can be satisfied by a citrate molecule. However, since the citrate molecule has three hydroxyl groups, two additional hydroxyl groups are available for coordination with other copper ions of adjacent ThiNO₃⁻(Cu)Gly molecules. If the configuration does not allow for all three hydroxyl sites to coordinate with three different Thi⁺NO₃⁻(Cu)Gly molecules, then water can be used to fill coordination sites as well The following is provided by way of example to illustrate a possible structure of the thiamine glycine chelate complex formed:

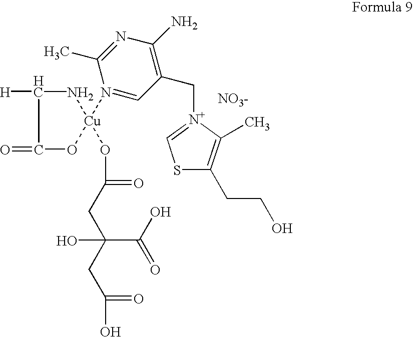

### Example 7

### Preparation of a Copper Glycine Thiamine Chelate Complex

A copper amino acid thiamine chelate complex was prepared by, first, reacting an equal molar mixture of copper hydroxide (48.8 g/L Cu(OH)₂), thiamine hydrochloride (168.7 g/L Thi⁺Cl⁻), and glycine (37.5 g/L Gly). Next, a source of acidic protons was added to help drive off the hydroxide ions (OH⁻) of the copper hydroxide. In the present example, acetic acid (Acetic) was added to the mixture. The following stoichiometry is provided to illustrate the reaction:

0.5Cu(OH)₂+0.5Thi⁺Cl⁻+0.5Gly+0.5Acetic

0.5Thi⁺Cl⁻(Cu)Gly+0.5Acetate+H₂O

In accordance with the above reaction scheme, the following procedures were followed to obtain about 267 g of total dissolved solids. Specifically, to 1 liter of water was added about 168.7 g of thiamine hydrochloride, and the mixture was brought to about 40° C. to 60° C. Next, about 37.5 g of glycine was added and the reaction mixture was allowed to react for from 3 to 5 minutes. About 28.6 g of acetic acid was then added to the reaction mixture and allowed to react for an additional 3 to 5 minutes. About 48.8 g of copper hydroxide was then added. After about 3 hours of further reaction, the product was spray dried at 40° C. About 267 g of product at about a 100% yield was produced.

It is believed that the coordination number of four was be fully satisfied. Specifically, two coordination sites can be satisfied by the glycine, one coordination site can be satisfied by the thiamine, and one coordination site can be satisfied by an acetate molecule. The following is provided by way of example to illustrate a possible structure of the thiamine amino acid chelate complex formed:

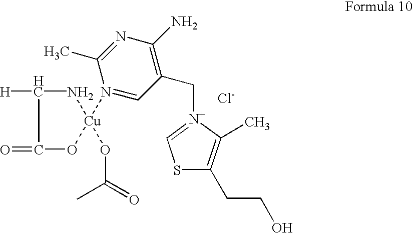

### Example 8

### Preparation of a Copper Glycine Thiamine Chelate Complex

A copper amino acid thiamine chelate complex was prepared by, first, reacting an equal molar mixture of copper hydroxide (48.8 g/L Cu(OH)₂), thiamine hydrochloride (168.7 g/L Thi⁺Cl⁻), and glycine (37.5 g/L Gly). Next, a source of acidic protons was added to help drive off the hydroxide ions (OH⁻) of the copper hydroxide. In the present example, citric acid (Citric) was added to the mixture. The following stoichiometry is provided to show the reaction:

0.5Cu(OH)₂+0.5Thi⁺Cl⁻+0.5Gly+0.25Citric

0.5Thi⁺Cl⁻(Cu)Gly+0.25Citrate+H₂O

In accordance with the above reaction scheme, the following procedures were followed to obtain about 287 g of total dissolved solids. Specifically, to 1 liter of water was added about 168.7 g of thiamine hydrochloride, and the mixture was brought to about 40° C. to 60° C. Next, about 37.5 g of glycine was added to reaction mixture and was allowed to further react for from 3 to 5 minutes. About 48.0 g of citric acid was then added to the reaction mixture and allowed to react for an additional 3 to 5 minutes. Next, about 48.8 g of copper hydroxide was added. After about 3 hours of further reaction, the product was spray dried at 40° C. About 287 g of product at about a 100% yield was produced.

It is believed that the coordination number of four can be fully satisfied. Specifically, two coordination sites can be satisfied by the glycine, one coordination site can be satisfied by the thiamine, and one coordination site can be satisfied by a citrate molecule. The following is provided by way of example to illustrate a possible structure of the thiamine amine acid chelate complex formed. However, other ancillary groups can be present other than the citrate group, or alternatively, the citrate group can act to satisfy a coordination number of a copper on an adjacent copper thiamine amino acid chelate complex. Thus, Formula 11 below provides one example of a possible structure:

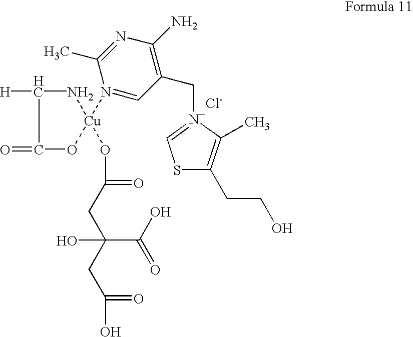

In Formula 11 above, two additional hydroxyl groups are present on the citrate ligand. Thus, either of those hydroxyl sites can also be used to fulfill the coordination site of an adjacent copper thiamine amino acid chelate complex molecule. In this example, as there are half as many moles of citric acid added compared to copper, thiamine, and glycine, each citrate can function to fill a coordination site of an adjacent metal of a thiamine amino acid chelate complex.

### Example 9

### Preparation of a Copper Glycine Thiamine Chelate Complex

A copper amino acid thiamine chelate complex in accordance with Formula 8 was prepared by, first, reacting an equal molar mixture of copper hydroxide (48.8 g/L Cu(OH)₂), thiamine mono-nitrate (163.7 g/L Thi⁺NO₃⁻), and glycine (37.5 g/L Gly). Next, a source of acidic protons was added to help drive off the hydroxide ions (OH⁻) of the copper hydroxide. In the present example, citric acid (Citric) was added to the mixture. The following stoichiometry is provided to illustrate the reaction:

0.5Cu(OH)₂+0.5Thi⁺NO₃⁻+0.5Gly+0.25Citric

0.5Thi⁺NO₃⁻(Cu)Gly+0.25Citrate+H₂O

In accordance with the above reaction scheme, the following procedures were followed to obtain about 279 g of total dissolved solids. Specifically, to i liter of water was added about 163.7 g of thiamine mono-nitrate, and the mixture was brought to about 40° C. to 60° C. Next, about 37.5 g of glycine was added to reaction mixture and the reaction mixture was allowed to react for from 3 to 5 minutes. About 48.0 g of citric acid was added to the reaction mixture and allowed to react for an additional 3 to 5 minutes. About 48.8 g of copper hydroxide was then added to the reaction mixture. After about 3 hours of further reaction, the product was cooled to about 40° C. and spray dried. About 279 g of product at about a 100% yield was produced.

It is believed that the coordination number of four can be fully satisfied. Specifically, two coordination sites can be satisfied by the glycine, one coordination site can be satisfied by the thiamine, and one coordination site can be satisfied by a citrate molecule. The following is provided by way of example to illustrate a possible structure of the thiamine glycine chelate complex formed. However, other ancillary groups can be present other than the citrate group shown, e.g., water, nitrate, etc. Alternatively, the citrate group can act to satisfy a coordination number of a copper on an adjacent copper thiamine amino acid chelate complex. Thus, Formula 12 below provides one example of a possible structure:

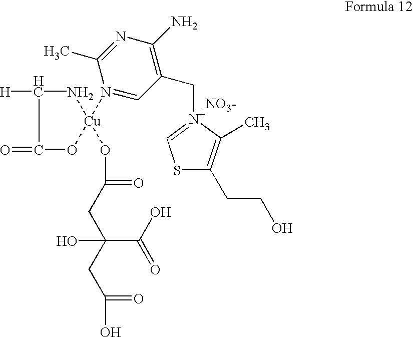

### Example 10

### Preparation of a Zinc Glycine Thiamine Chelate Complex

A zinc amino acid thiamine chelate complex was prepared by, first, reacting an equal molar mixture of zinc oxide (40.7 g/L ZnO), thiamine mono-nitrate (163.7 g/L Thi⁺NO₃⁻), and glycine (37.5 g/L Gly). Next, a source of acidic protons was added to help drive off the hydroxide ions (OH⁻) of the hydrogenated zinc oxide. In the present example, citric acid (Citric) was added to the mixture. The following stoichiometry is provided to illustrate the reaction:

0.5ZnO+0.5Thi⁺NO₃⁻+0.5Gly+0.25Citric

0.5Thi⁺NO₃⁻(Zn)Gly+0.25Citrate+H₂O

In accordance with the above reaction scheme, the following procedures were followed to obtain about 279 g of total dissolved solids. Specifically, to 1 liter of water was added about 163.7 g of thiamine mono-nitrate, and the mixture was brought to about 40° C. to 60° C. Next, about 37.5 g of glycine was added and the reaction mixture was allowed to progress for from 3 to 5 minutes. About 48.0 g of citric acid was then added and allowed to react for an additional 3 to 5 minutes. About 40.7 g of zinc oxide was then added to the mixture. After about 3 hours of further reaction, the product was spray dried to 40° C. About 279 g of product at about a 100% yield was produced.

It is believed that the coordination number of four can be fully satisfied. Specifically, two coordination sites can be satisfied by the glycine, one coordination site can be satisfied by the thiamine, and one coordination site can be satisfied by a citrate molecule. However, the citrate can act to satisfy the coordination number of two or three separate zinc ions from two or three separate thiamine amino acid chelate complexes. The following is provided by way of example to illustrate a possible structure of the thiamine glycine chelate complex formed:

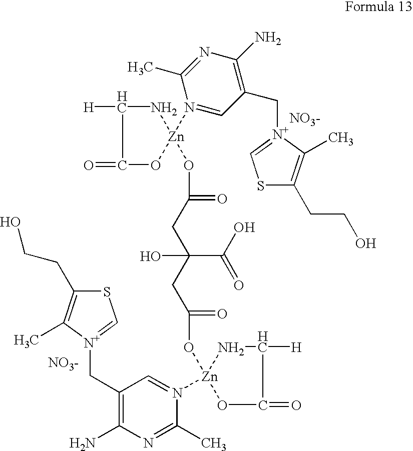

### Example 11

### Preparation of a Manganese Serine Thiamine Chelate Complex

A manganese amino acid thiamine chelate complex was prepared by, first, reacting an equal molar mixture of manganese oxide (35.5 g/L MnO), thiamine mono-nitrate (163.7 g/L Thi⁺NO₃⁻), and serine (52.6 g/L Ser). Next, a source of acidic protons was added to help drive off the hydroxide ions (OH⁻) of the hydrogenated zinc oxide. In the present example, maleic acid (Maleic) was added to the mixture. The following stoichiometry is provided to illustrate the reaction:

0.5MnO+0.5Thi⁺NO₃⁻+0.5Ser+0.25Maleic

0.5Thi⁺NO₃⁻(Mn)Ser+0.25Maleate+0.5H₂O

In accordance with the above reaction scheme, the following procedures were followed to obtain about 270 g of total dissolved solids. Specifically, to 1 liter of water was added about 163.7 g of thiamine mono-nitrate, and the mixture was brought to about 40° C. to 60° C. Next, about 52.6 g of serine was added and the reaction mixture was allowed to react for from 3 to 5 minutes. About 29.0 g of maleic acid was then added and allowed to react for an additional 3 to 5 minutes, followed by the addition of about 35.5 g of manganese oxide. After about 3 hours of 5 further reaction, the product was spray dried at 40° C. About 270 g of product at about a 100% yield was produced.

It is believed that the coordination number of four will likely be fully satisfied. Specifically, two coordination sites can be satisfied by the serine, one coordination site can be satisfied by the thiamine, and one coordination site can be satisfied by a maleate molecule. However, the maleate can act to satisfy the coordination number of two separate manganese ions from two separate thiamine amino acid chelate complexes. The following is provided by way of example to illustrate a possible structure of the thiamine serine chelate complex formed:

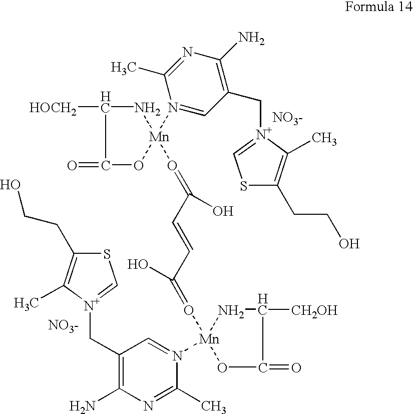

### Example 12

### Preparation of a Manganese Cysteine Thiamine Chelate Complex

A manganese amino acid thiamine chelate complex was prepared by, first, reacting an equal molar mixture of manganese oxide (35.5 g/L MnO), thiamine hydrochloride (168.7 g/L Thi⁺Cl⁻), and cysteine (60.6 g/L Cys). Next, a source of acidic protons was added to help drive off the hydroxide ions (OH⁻) formed when the manganese oxide is hydrogenated. In the present example, acetic acid (Acetic) was added to the mixture. The following stoichiometry is provided to illustrate the reaction:

0.5MnO+0.5Thi⁺Cl⁻+0.5Cys+0.5Acetic

0.5Thi⁺Cl⁻(Mn)Cys+0.5Acetate+H₂O

In accordance with the above reaction scheme, the following procedures were followed to obtain about 270 g of total dissolved solids. Specifically, to 1 liter of water was added about 168.7 g of thiamine hydrochloride, and the mixture was brought to about 40° C. to 60° C. Next, about 60.6 g of cysteine was added to reaction mixture and the reaction mixture was allowed to react for from 3 to 5 minutes. About 28.6 g of acetic acid was added to the reaction mixture and allowed to react for an additional 3 to 5 minutes. About 35.5 g of manganese oxide was then added followed by about 3 hours of further reaction time. The product was spray dried at 40° C. About 270 g of product at about a 100% yield was produced.

It is believed that the coordination number of four can be fully satisfied. Specifically, two coordination sites can be satisfied by the cysteine, one coordination site can be satisfied by the thiamine, and one coordination site can be satisfied by an acetate molecule. The following is provided by way of example to illustrate a possible structure of the thiamine cysteine chelate complex formed:

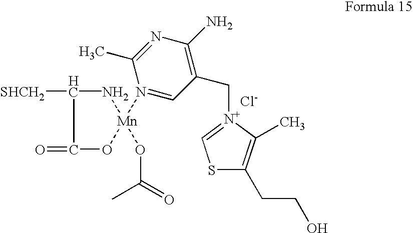

### Example 13

### Preparation of a Zinc Glycine Thiamine Chelate Complex

A zinc amino acid thiamine chelate complex was prepared by, first, reacting an equal molar mixture of zinc oxide (40.7 g/L ZnO), thiamine hydrochloride (168.7 g/L Thi⁺Cl⁻), and glycine (37.5 g/L Gly). Next a source of acidic protons was added to help drive off the hydroxide ions (OH⁻) once the zinc oxide is hydrogenated. In the present example, acetic acid (Acetic) was added to the mixture. The following stoichiometry is provided to show the reaction:

0.5ZnO+0.5Thi⁺Cl⁻+0.5Gly+0.5Acetic

0.5Thi⁺Cl⁻(Zn)Gly+0.5Acetate+H₂O

In accordance with the above reaction scheme, the following procedures were followed to obtain about 269 g of total dissolved solids. Specifically, to 1 liter of water was added about 168.7 g of thiamine hydrochloride, and the mixture was brought to about 40° C. to 60° C. Next, about 37.5 g of glycine was added to reaction mixture and the reaction mixture was allowed to react for from 3 to 5 minutes. About 28.6 g of acetic acid was added and allowed to react for an additional 3 to 5 minutes. About 40.7 g of zinc oxide was then added to the reaction mixture. After about 3 hours of further reaction, the product was spray dried at 40° C. About 269 g at about a 100% yield was produced.

It is believed that the coordination number of four can be fully satisfied. Specifically, two coordination sites can be satisfied by the glycine, one coordination site can be satisfied by the thiamine, and one coordination site can be satisfied by an acetate molecule. The following is provided by way of example to illustrate a possible structure of the thiamine glycine chelate complex formed:

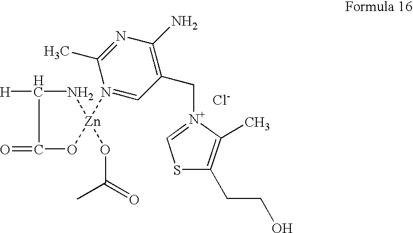

### Example 14

### Preparation of a Manganese Glycine Thiamine Chelate Complex

A manganese amino acid thiamine chelate complex was prepared by, first, reacting an equal molar mixture of manganese carbonate (57.4 g/L MnCO₃), thiamine mono-nitrate (163.7 g/L Thi⁺NO₃⁻), and glycine (37.5 g/L Gly). Next, a source of acidic protons was added to help drive off the carbonate ion (CO₃⁻) of the manganese carbonate. In the present example, citric acid (Citric) was added to the mixture. The following stoichiometry is provided to illustrate the reaction:

0.5MnCO₃+0.5Thi⁺NO₃⁻+0.5Gly+0.25Citric

0.5Thi⁺NO₃⁻(Mn)Gly+0.5CO₂+0.25Citrate+0.5H₂O

In accordance with the above reaction scheme, the following procedures were followed to obtain about 270 g of total dissolved solids. Specifically, to 1 liter of water was added about 163.7 g of thiamine mono-nitrate, and the mixture was brought to about 40° C. to 60° C. Next, about 37.5 g of glycine was added and the mixture was allowed to react for from 3 to 5 minutes. About 48.0 g of citric acid was added and allowed to react for an additional 3 to 5 minutes. About 57.4 g of manganese carbonate was then added. After about 3 hours of further reaction, the product was spray dried at 40° C. About 279 g of product at about a 100% yield was produced.

It is believed that the coordination number of four can be fully satisfied. Specifically, two coordination sites can be satisfied by the glycine, one coordination site can be satisfied by the thiamine, and one coordination site can be satisfied by a citrate molecule. However, the citrate can act to satisfy the coordination number of two or three separate zinc ions from two or three separate thiamine amino acid chelate complexes. The following is provided by way of example to illustrate a possible structure of the thiamine glycine chelate complex formed:

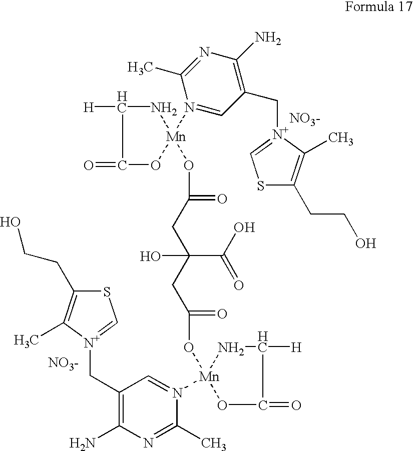

### Example 15

### Preparation of a Zinc Glycine Thiamine π-Bond Chelate Complex

To a mixture of 0.34 g (2.5 mmole) of ZnCl₂ in 0.34 g of water was slowly added 0.24 g (2.5 mmole) of sodium glycinate and 0.84 g (2.5 mmole) of thiamine hydrochloride in 1.5 g of water. About 0.06 ml of H₃PO₄ was added and the mixture was heated to 70° C. where it became a clear solution. The solution was washed with ethanol and no precipitate formed. The solution was placed in a refrigerator overnight and was then placed on a workbench at room temperature. Within an hour after being removed from the refrigerator, crystals precipitated from the solution. The crystals have a melting point of about 145° C. to 155° C., a solubility of about 10 mg/ml. The pH of a saturated solution was about 2.4. When ZnCl₂ was replaced with 2.5 mm (0.40 g) of ZnSO₄ and the same procedure was followed, the recrystallized product melted between about 200° and 205° C., had about the same solubility but had a pH of about 2.9. There was obviously some difference caused in either the purity or the structure resulting from the use of a chloride salt as compared to a sulfate. However, IR spectra showed that chelation had occurred. The chelate formed is believed to have π-cloud structure as shown below in Formula 18:

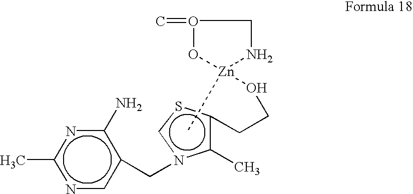

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method for reducing alcohol desire or dependency, comprising the steps of:
administering a therapeutically effective amount of an amino acid chelate to a human having symptoms of alcohol desire or dependency, said amino acid chelate comprising a naturally occurring amino acid ligand and a metal selected from the group consisting of copper, zinc, and manganese, and wherein the amino acid to metal molar ratio is from 1:1 to 4:1.

2. A method as in claim 1, wherein the metal is copper.

3. A method as in claim 1, wherein the metal is zinc.

4. A method as in claim 1, wherein the metal is manganese.

5. A method as in claim 1, further comprising coadministering to the human a second amino acid chelate that is different than the amino acid chelate, said second amino acid chelate also comprising a naturally occurring amino acid ligand and a metal selected from the group consisting of copper, zinc, and manganese, and wherein the second amino acid chelate has an amino acid to metal molar ratio from 1:1 to 4:1.

6. A method as in claim 1, further comprising coadministering to the human a therapeutically effective amount of thiamine or a thiamine-containing composition.

7. A method as in claim 6, wherein the thiamine-containing composition is coadministered and comprises thiamine or a thiamine phosphate molecule which is chelated or complexed to a nutritionally relevant metal.

8. A method as in claim 1, further comprising coadministering to the human a therapeutically effective amount of a metal carnitine chelate, said metal carnitine chelate comprising a metal selected from the group consisting of copper, zinc, or manganese, and wherein the carnitine to metal molar ratio is from 1:1 to 2:1.

9. A method as in claim 1, wherein the amino acid is selected from the group consisting of glycine, cystine, cysteine, arginine, histidine, lysine, glutamic acid, and combinations thereof.

10. A method as in claim 5, further comprising coadministering to the human a third amino acid chelate that is different than the amino acid chelate and the second amino acid chelate, said third amino acid chelate also comprising a naturally occurring amino acid ligand and a metal selected from the group consisting of copper, zinc, and manganese, and wherein the third amino acid chelate has an amino acid to metal molar ratio from 1:1 to 4:1.

11. A method for reducing alcohol desire or dependency, comprising the steps of:
administering a therapeutically effective amount of a composition to a human having symptoms of alcohol desire or dependency, said composition comprising a naturally occurring amino acid chelated to a nutritionally relevant metal, said composition further comprising thiamine complexed to the nutritionally relevant metal.

12. A method as in claim 11, wherein the nutritionally relevant metal is selected from the group consisting of calcium, magnesium, copper, zinc, and manganese.

13. A method as in claim 11, wherein the thiamine is a thiamine phosphate, and the thiamine phosphate is completed at its phosphate moiety to the nutritionally relevant metal.

14. A method as in claim 11, wherein the thiamine is complexed at the N¹ position of its pyrimidine ring to the nutritionally relevant metal.

15. A method as in claim 11, further comprising coadministering to the human a therapeutically effective amount of an amino acid chelate comprising a naturally occurring amino acid ligand and a metal selected from the group consisting of copper, zinc, and manganese, wherein the amino acid to metal molar ratio is from 1:1 to 4:1.

16. A method as in claim 11, further comprising coadministering to the human a therapeutically effective amount of thiamine or a second thiamine-containing composition.

17. A method as in claim 1, further comprising coadministering to the human a therapeutically effective amount of a carnitine ligand chelated to a metal selected from the group consisting of copper, zinc, or manganese, wherein the carnitine to metal molar ratio is from 1:1 to 2:1.

18. A method as in claim 11, wherein the naturally occurring amino acid is selected from the group consisting of glycine, cystine, cysteine, arginine, histidine, lysine, glutamic acid, and combinations thereof.

19. A method as in claim 11, wherein a coordination number of the metal is fully satisfied.

20. A method as in claim 11, further comprising an ancillary molecule complexed or coordinated to the metal.

21. A method for reducing alcohol desire or dependency, comprising the steps of:
administering a therapeutically effective amount of a metal carnitine chelate to a human having symptoms of alcohol desire or dependency, said metal carnitine chelate comprising a metal selected from the group consisting of copper, zinc, or manganese, and wherein the carnitine to metal molar ratio is from 1:1 to 2:1.

22. A method as in claim 21, wherein the metal is copper.

23. A method as in claim 21, wherein the metal is zinc.

24. A method as in claim 21, wherein the metal is manganese.

25. A method as in claim 21, further comprising coadministering to the human an amino acid chelate, said amino acid chelate comprising a naturally occurring amino acid ligand and a metal selected from the group consisting of copper, zinc, and manganese, and wherein the second amino acid to metal molar ratio is from 1:1 to 4:1.

26. A method as in claim 21, further comprising coadministering to the human a therapeutically effective amount of thiamine or a thiamine-containing composition.

27. A method as in claim 26, wherein the thiamine-containing composition is coadministered and comprises thiamine or a thiamine phosphate molecule which is chelated or complexed to a nutritionally relevant metal.

28. A method for reducing alcohol desire or dependency, comprising the steps of:
administering a therapeutically effective amount of a metal thiamine chelate to a human having symptoms of alcohol desire or dependency, wherein the metal is chelated to thiamine through a π-cloud of an aromatic ring.

29. A method as in claim 28, wherein a naturally occurring amino acid is also chelated to the metal.

30. A composition for reducing alcohol dependency, comprising a metal carnitine chelate, said metal being selected from the group consisting of copper, zinc, or manganese, wherein the carnitine to metal molar ratio is from 1:1 to 2:1.

31. A composition as in claim 30, further comprising an amino acid chelate admixed or blended with the metal carnitine chelate, said amino acid chelate comprising a naturally occurring amino acid ligand and a metal selected from the group consisting of copper, zinc, and manganese, and wherein the amino acid to metal molar ratio is from 1:1 to 4:1.

32. A composition as in claim 30, further comprising thiamine or a thiamine-containing composition admixed or blended with the metal carnitine chelate.

33. A thiamine-complexed metal amino acid chelate composition for reducing alcohol dependency, comprising a naturally occurring amino acid chelated to a nutritionally relevant metal, said composition further comprising thiamine complexed to the nutritionally relevant metal.

34. A composition as in claim 33, wherein the thiamine is a thiamine phosphate, and the thiamine phosphate is complexed to the metal at its phosphate moiety.

35. A composition as in claim 33, wherein the thiamine is complexed to the metal at the N¹ position of its pyrimidine ring.

36. A composition as in claim 33, wherein the nutritionally relevant metal is selected from the group consisting of calcium, magnesium, copper, zinc, and manganese.

37. A composition as in claim 33, wherein the naturally occurring amino acid is selected from the group consisting of glycine, cystine, cysteine, arginine, histidine, lysine, glutamic acid, and combinations thereof.

38. A composition as in claim 33, wherein the amino acid to metal molar ratio is about 1:1.

39. A composition as in claim 33, wherein the thiamine to metal molar ratio is about 1:1.

40. A composition as in claim 33, wherein the thiamine to amino acid to metal molar ratio is about 1:1:1.

41. A composition as in claim 33, wherein the amino acid to metal molar ratio is about 2:1.

42. A composition as in claim 33, wherein the thiamine to metal molar ratio is about 2:1.

43. A composition as in claim 33, wherein a coordination number of the metal is fully satisfied.

44. A composition as in claim 33, further comprising an ancillary molecule complexed or coordinated to the metal.

45. A composition as in claim 44, wherein the ancillary molecule is selected from the group consisting of water, acids, chloride, and nitrates.

46. A composition as in claim 33, wherein the thiamine is a thiamine phosphate selected from the group consisting of thiamine mono-phosphate, thiamine di-phosphate, and thiamine tri-phosphate.

47. A composition for reducing alcohol dependency in humans, comprising a particulate blend of:
a) a first amino acid chelate comprising a naturally occurring amino acid and a metal selected from the group consisting of copper, zinc, and manganese; and
b) a second amino acid chelate comprising a second naturally occurring amino acid and
a second metal selected from the group consisting of copper, zinc, and manganese,
wherein the first amino acid chelate and the second amino acid chelate each having an amino acid to metal molar ratio from 1:1 to 4:1, and wherein the first amino acid chelate and the second amino acid chelate each have a different metal.

48. A composition as in claim 47, wherein the first amino acid chelate is a copper amino acid chelate, the second amino acid chelate is a zinc amino acid chelate, and wherein the particulate blend further comprises a third amino acid chelate comprising a naturally occurring amino acid and manganese, said third amino acid chelate having an amino acid to metal molar ratio from 1:1 to 4:1.
